(19) United States
(12) Patent Application Publication
(10) Pub. No.: US 2007/0244400 A1
(43) Pub. Date: Oct. 18, 2007

(54) Compliant biometric sensor apparatus

(76) Inventor: Ed Honda, Chicago, IL (US)

Correspondence Address: HANLEY, FLIGHT & ZIMMERMAN, LLC, 150 S. WACKER DRIVE, SUITE 2100, CHICAGO, IL, 60606, US

(21) Appl. No.: 11/296,761

(22) Filed: Dec. 7, 2005

(51) Int. Cl. A61B 5/04

(52) U.S. Cl. 600/509

(57) ABSTRACT

Compliant biometric sensor apparatus are disclosed. A disclosed example biometric sensor apparatus includes a handgrip having a length and at least one substantially compliant portion extending along at least a portion of the length and at least one electrode coupled to the compliant portion so that the electrode is movable relative to at least a portion of the handgrip.

### FIELD OF THE DISCLOSURE

The present disclosure relates generally to biometric sensors and, more particularly, to compliant biometric sensor apparatus and systems including biometric sensor apparatus.

### BACKGROUND

Modern exercise machine technology enables providing users with immediate performance feedback information. Performance feedback information includes exercise machine operation information such as speed, incline, repetitions, resistance, elapsed/remaining workout time, etc. Although such machine operation information provides a guide for measuring improvements in the amount of work that a user can do, the actual health or physiological improvement or status of a user is typically monitored by measuring the user's physiological signals or vital signals such as heart rate.

To measure a person's physiological condition such as, for example, a person's cardiovascular condition, laboratories and/or physical fitness facilities often use expensive, complex instrumentation that employs transducer pads adhered to a person's skin and wired to biometric instrumentation. In this manner, a person's heart rate can be measured while the person exercises on a stationary exercise machine such as a treadmill, a stepper machine, an elliptical cross-trainer machine, a weight-training machine, etc. Measuring a person's physiological condition in this manner can be relatively expensive, and availability of such equipment is often limited to career athletes, rehabilitation patients, and/or members of expensive fitness programs or clubs.

Many physical fitness equipment companies develop technologies to make physiological feedback more accessible to anyone owning an exercise machine or a fitness club membership. In particular, some fitness companies design and manufacture handgrip sensors that measure a person's heart rate by detecting physiological signals through the person's hands. Such known biometric sensor handgrips implemented on exercise machines typically include a conductive plate (i.e., a biometric electrode) mounted rigidly onto a handgrip, a handrail, or a handlebar that enables a person to wrap their hand about a surface of the conductive plate as the person grips the handgrip, handrail, or handlebar. As the person exercises, the conductive plate or biometric electrode senses physiological signals (i.e., electrical signals) that emanate through the person's skin. The physiological signals are then communicated to a processor and/or hardware system that performs signal-processing operations to determine the person's heart rate.

Known handrail or handgrip biometric electrodes are typically susceptible to sensing a lot of electrical noise while sensing physiological signals. Although some of the electrical noise is due to aliasing or echoes associated with the physiological signals, other electrical noises are due to the poor coupling between the biometric electrodes and a person's hands. Exercise equipment manufacturers often rely on signal processing operations to filter out aliasing and echo. In many instances, filtering out aliasing and echo using signal-processing algorithms can be relatively easy because of their characteristic periodicity. However, more difficult signals to filter out using signal-processing operations are those having a periodic or sporadic characteristics such as noises associated with minor engagement and disengagement between a person's hands and the biometric sensors. For example, while exercising, a person's arms and hands can be subject to a lot of movement such that the amount of contact or engagement between their hands and the biometric sensors changes frequently. Even the slightest engagement and disengagement, which may be imperceptible to the person, can produce large amounts of electrical noise. In many instances, electrical noise that cannot be successfully filtered out by signal processing operations becomes averaged or incorporated into the determination of a person's heart rate, thus leading to inaccurate physiological performance feedback. In some instances, the inability to or difficulty in filtering out electrical noise may lead to poor system response time in determining and providing feedback to a user.

### BRIEF DESCRIPTION OF THE DRAWINGS

FIG. 1 is a detailed depiction of an example compliant biometric sensor handgrip.

FIG. 2 is an exploded isometric view of the example compliant biometric sensor handgrip depicted in FIG. 1.

FIG. 3 is an isometric view of the compliant electrode mounts used to form the example compliant biometric sensor handgrip of FIGS. 1 and 2.

FIG. 4 depicts an example manner in which a person's hand may grip the example compliant biometric sensor handgrip of FIGS. 1 and 2 and apply a plurality of forces to biometric electrodes of the example compliant biometric sensor handgrip.

FIGS. 5-7 illustrate representative cross-sectional views of the example compliant biometric sensor handgrip of FIGS. 1, 2, and 4 depicting by way of example some of the manners in which portions of the compliant electrode mounts of FIG. 3 become deformed or displaced in response to some of the forces indicated in FIG. 4.

FIG. 8 is an example exercise machine having handgrips implemented using the example compliant biometric sensor handgrip depicted in FIGS. 1, 2, and 4.

FIG. 9 is another example exercise machine having handrails implemented using a compliant biometric sensor apparatus.

### DETAILED DESCRIPTION

Although the following discloses example apparatus, systems, and articles of manufacture, it should be noted that such apparatus, systems, and articles of manufacture are merely illustrative and should not be considered as limiting. For example, it is contemplated that while the following describes example apparatus, systems, and articles of manufacture, persons of ordinary skill in the art will readily appreciate that the examples provided are not the only way to implement such apparatus, systems, and articles of manufacture.

The example compliant biometric sensor apparatus described herein may be used to measure, for example, a person's heart rate while the person exercises. Unlike known configurations of biometric electrodes rigidly mounted on exercise machines, the example compliant biometric sensor apparatus described herein are implemented by mounting movable or floating biometric electrodes on exercise machines to enable relatively better contact (e.g., more consistent engagement) between a person's hand and the biometric electrode during exercise.

During exercise a person's body produces a lot of motion, and a person's hand gripping a biometric electrode rigidly mounted on an exercise machine often slides or slips along portions of the biometric electrode, and/or the amount of surface area of a person's hand (i.e., contact area) engaging the biometric electrode continuously changes. While sensing a person's physiological signals, the biometric electrode generates noise or is unable to accurately detect the physiological signals any time the person's hand slips along the biometric electrode surface and/or as the amount of contact area between the person's hand and the electrode changes. To improve the physiological information produced by an exercise machine, the apparatus, systems, and articles of manufacture described herein may be used to compliantly mount a biometric electrode to an exercise machine to maintain better hand-to-electrode contact during exercise. Specifically, the compliant biometric sensor apparatus described herein enables the amount of contact area between a person's hand and a biometric electrode to be more consistent such that the contact area substantially unaffected by a person's typical range of motions during exercise.

As described in greater detail below, an example compliant biometric sensor apparatus may be implemented using a handgrip having a compliant material portion, element, or member extending along a portion of the handgrip and coupling an electrode to the compliant material portion, element or member so that the electrode is movable relative to at least a portion of the handgrip as a person grips the electrode and the handgrip during exercise. For example, the handgrip may be implemented using a handgrip base having a particular hardness characteristic (e.g., a relatively high (70-80) Shore D durometer hardness). The compliant portion may be used to form an electrode mount coupled to the handgrip base and may have a relatively softer or lower hardness characteristic (e.g., a relatively low (30-35) Shore D durometer hardness) that is substantially different from the hardness of the handgrip base. These relatively different hardness characteristics enable differential movement between opposing surfaces of the electrode mount so that an electrode may be movably coupled to the electrode mount.

The relatively lower hardness of the electrode mount enables the electrode to be substantially movable relative to the handgrip base. In this manner, as a person exercises and the person's arms and hands move relative to the handgrip base, the person's hand grip on the electrode can remain relatively firm and consistent. In some example implementations, the electrode mount may be implemented using a relatively pliable material (e.g., a rubber material, a gel pad, a low-durometer thermoplastic elastomer, etc.) that enables the electrode to float on (e.g., to be displaceable or movable relative to) the electrode mount and relative to the handgrip base.

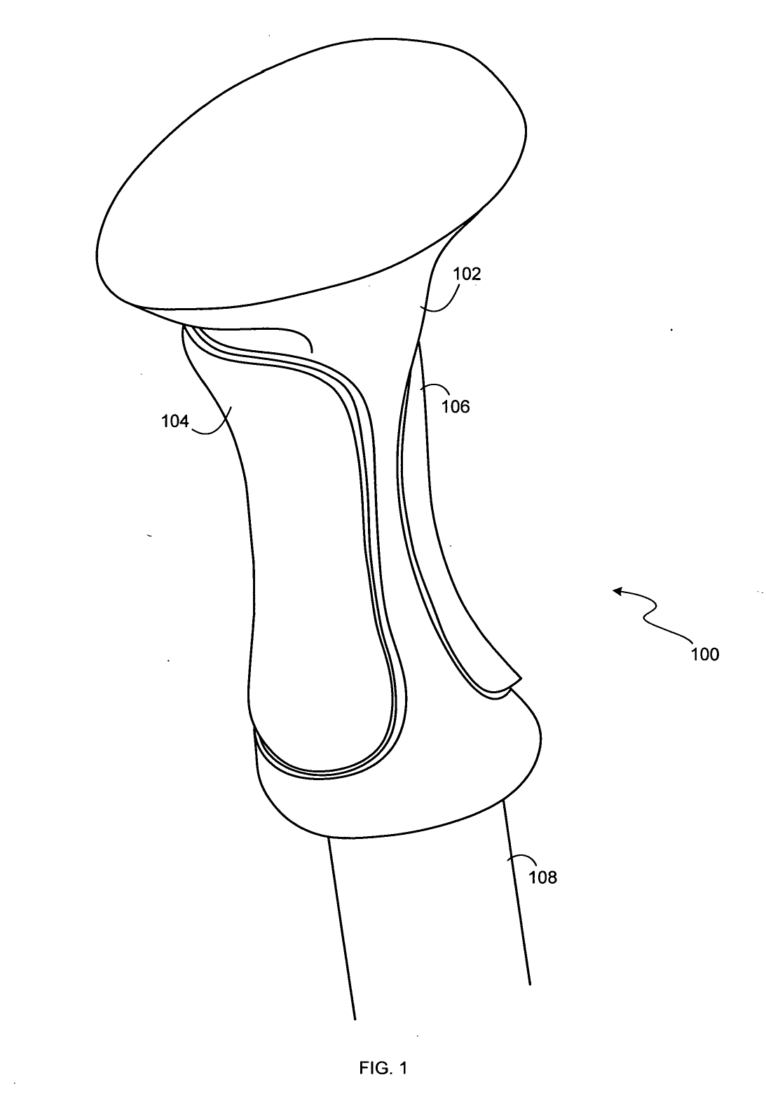

Now turning in detail to FIG. 1, an example compliant biometric sensor handgrip 100 includes a handgrip base 102, a first electrode 104 (e.g., a conductive element) mounted to a first portion of the handgrip base 102, and a second electrode 106 (e.g., another conductive element) mounted on a second portion of the handgrip base 102 opposing the first electrode 104. Although in the illustrated example, the electrodes 104 and 106 are shown as mounted in an opposing configuration, in other example implementations, electrodes may be mounted adjacent one another or in other suitable configurations. Also, other example implementations may include fewer or more electrodes.

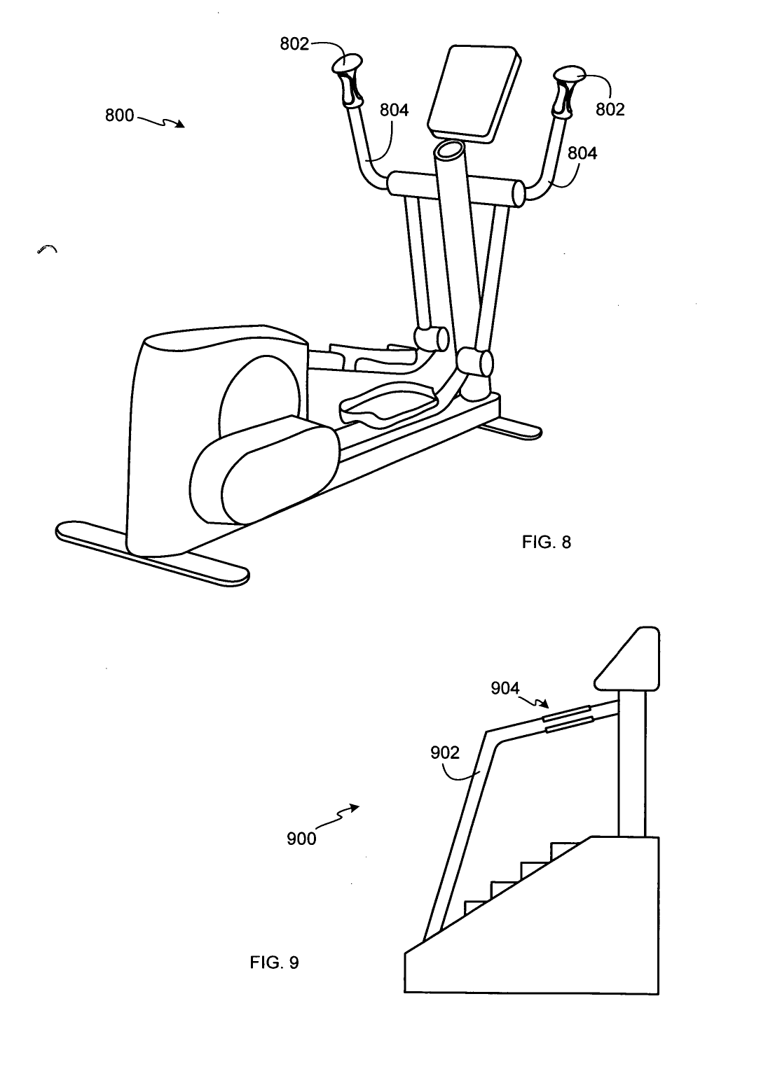

As shown in FIG. 1, the handgrip base 102 is a sleeve-like structure or member that may be mounted onto a tubular-like member or bar 108, which may form part of an exercise machine such as an elliptical cross-trainer machine (e.g., the elliptical cross-trainer machine 800 of FIG. 8), a stair-stepper machine (e.g., the stair-stepper machine 900 of FIG. 9), a stationary bicycle machine (not shown), a stationary recumbent bicycle machine (not shown), etc. Although the example compliant biometric sensor apparatus (e.g. the example compliant biometric sensor handgrip 100) are described herein with respect to stationary exercise machines, the example compliant biometric sensor apparatus may be implemented in combination with non-stationary machines or apparatus. For example, the example compliant biometric sensor handgrip 100 may be mounted to a non-stationary bicycle and used to measure a person's heart rate as the person rides outdoors.

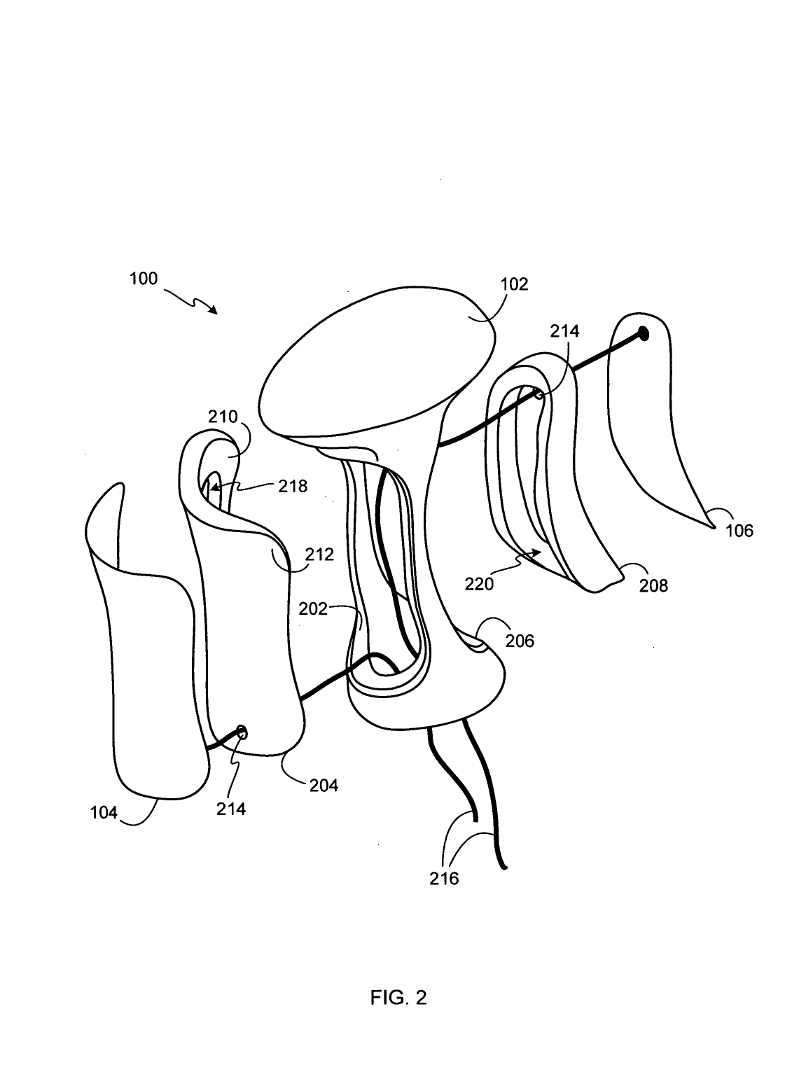

FIG. 2 depicts an exploded isometric view of the example compliant biometric sensor handgrip 100 depicted in FIG. 1. As shown, the handgrip base 102 includes a first handgrip base surface 202 that is configured to receive a first compliant electrode mount 204 and a second handgrip base surface 206 opposing the first handgrip base surface 202 that is configured to receive a second compliant electrode mount 208. The first compliant electrode mount 204 includes an inner electrode mount surface 210 that engages the first handgrip base surface 202 when the first compliant electrode mount 204 is coupled to the handgrip base 102. The first compliant electrode mount 204 also includes an outer electrode mount surface 212 that receives the first electrode 104 such that the first electrode 104 is in engagement with the outer electrode mount surface 212 when the first electrode 104 is coupled to the first compliant electrode mount 204.

In some example implementations, the first compliant electrode mount 204 may be coupled to the handgrip base 102 and the first electrode 104 may be coupled to the first compliant electrode mount 204 using a liquid adhesive and/or an adhesive tape. In some example implementations, the compliant electrode mounts 204 and 208 may be injection molded directly onto the electrodes 104 and 106 to avoid using adhesive between the compliant electrode mounts 204 and 208 and the respective electrodes 104 and 106.

The second compliant electrode mount 208 may be coupled to the handgrip base 102 via the second handgrip base surface 206 in substantially the same manner as the first compliant electrode mount 204 is coupled to the handgrip base 102. Also, the second electrode 106 may be coupled to the second compliant electrode mount 208 in substantially the same manner as the first electrode 104 is coupled to the first compliant electrode mount 204.

A hole or aperture 214 may be formed in each of the compliant electrode mounts 204 and 208 to insert signal conductors 216 (e.g., wires) therethrough that are used to communicate signals representing physiological signals detected via the electrodes 104 and 106 to a processing device (e.g., a heart rate monitor) (not shown).

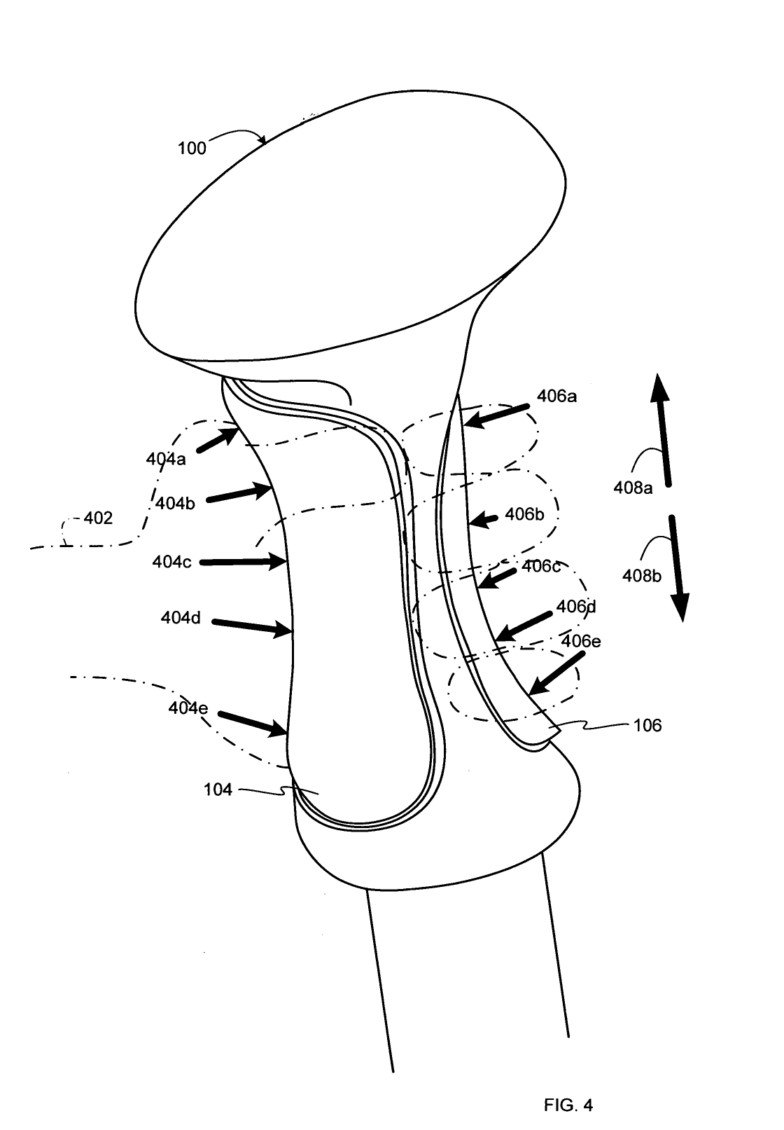

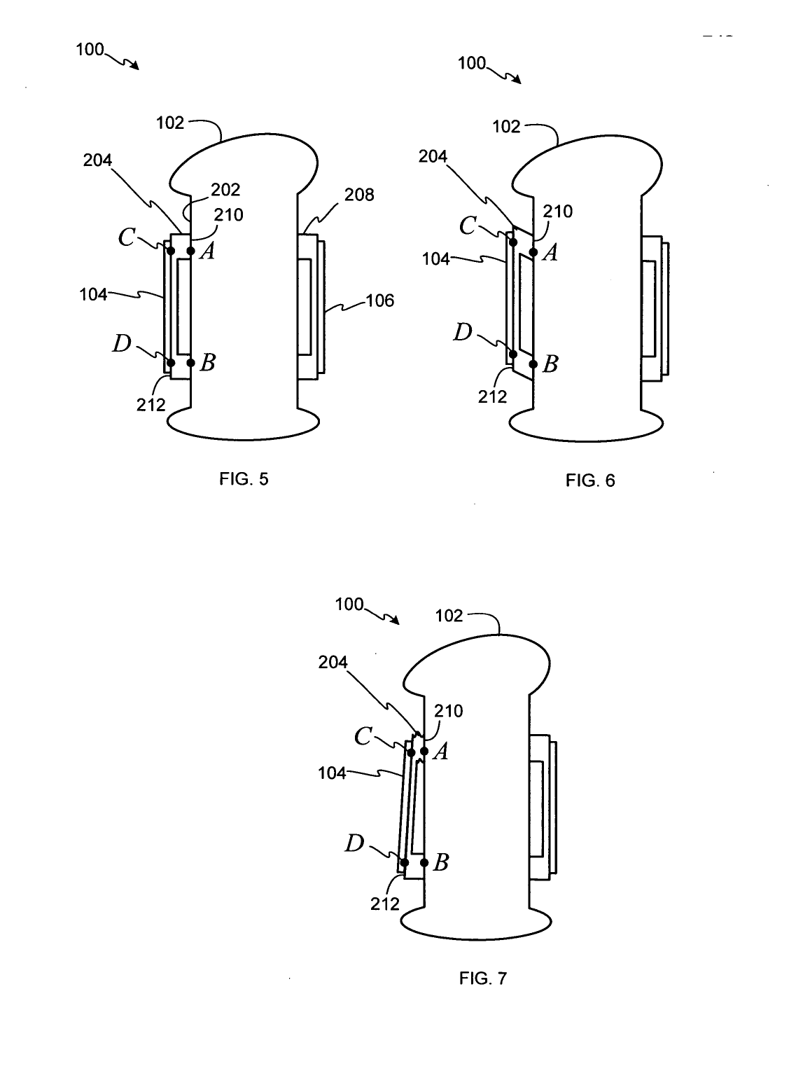

The compliant electrode mounts 204 and 208 may be implemented using a relatively pliable or compliant material (e.g., a rubber material, a gel pad, a low-durometer thermoplastic elastomer, etc.). In this manner, when forces are applied to the compliant electrode mounts 204 and 208 via the electrodes 104 and 106 as described below in connection with FIG. 4, portions of the pliable or compliant material displace relative to other portions as described below in connection with FIGS. 5-7 to enable substantially consistent contact or engagement between a person's hand (e.g., the hand 402 shown in FIG. 4) and the electrodes 104 and 106. Example materials that may be used to implement the compliant electrode mounts 204 and 206 include rubber, polyurethane, thermoplastic elastomers, gel, foam, etc. In some example implementations, the compliant electrode mounts 204 and 206 may also be implemented using springs or spring arrays (e.g., a pad having an array or grid of relatively small springs). In any case, the compliant electrode mounts 204 and 208 should be implemented to enable differential movement between the inner compliant electrode mount surface 210 and the outer compliant electrode mount surface 212 as described below in connection with FIGS. 5-7 so that the electrodes 104 and 106 are movable relative to the handgrip base 102.

To provide support for the compliant electrode mounts 204 and 208, the material used to implement the handgrip base 102 may be selected to be relatively harder than the material used to implement the compliant electrode mounts 204 and 208. For example, the compliant electrode mounts 204 and 208 may be implemented using a material of about 30-35 Shore D durometer hardness (e.g., a low-durometer thermoplastic elastomer) and the handgrip base 102 may be implemented using a material of about 70-80 Shore D durometer hardness (e.g., a high-durometer thermoplastic elastomer).

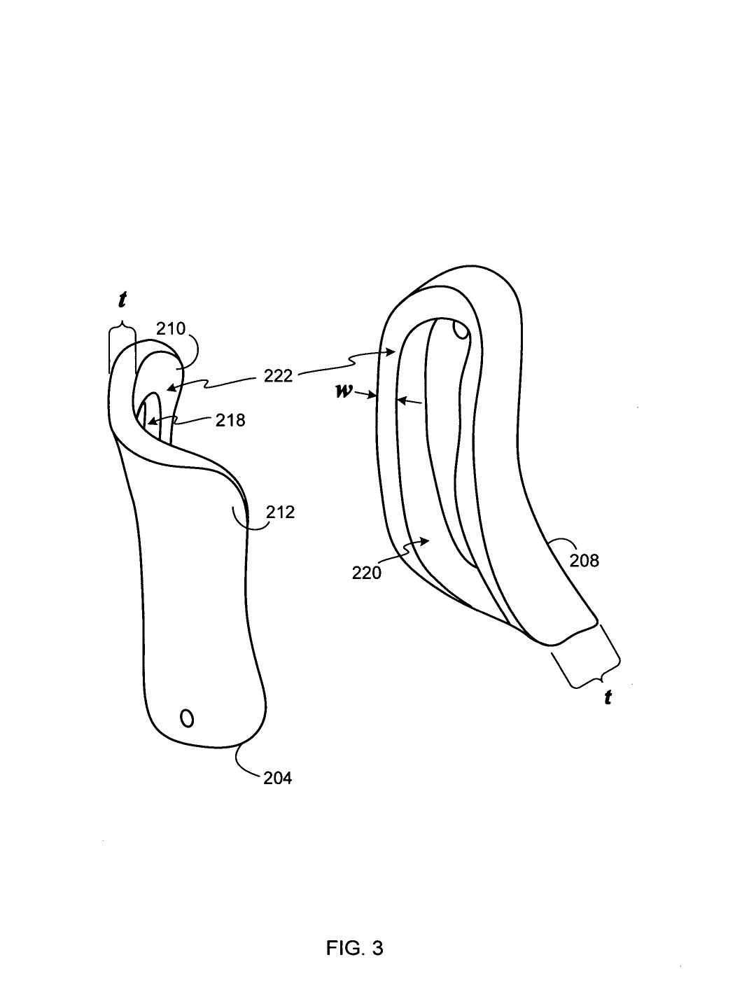

The thickness (e.g., the thicknesses t shown in FIG. 3) of the compliant electrode mounts 204 and 208 may be selected as a function of the material durometer (e.g., the material hardness) and the desired level of compliance (e.g., the amount of differential movement required or desired between the inner compliant electrode mount surface 210 and the outer compliant electrode mount surface 212). Also, the thickness t of the compliant electrode mounts 204 and 208 may be selected so that the electrodes 104 and 106, when coupled to the compliant electrode mounts 204 and 208, do not engage or are substantially out of contact with or separated from the handgrip base 102. In this manner, the movement of the electrodes 104 and 106 relative to the handgrip base 102 is substantially uninhibited by the handgrip base 102 as the electrodes 104 and 106 float on the compliant electrode mounts 204 and 208. In addition, the thickness t of the first compliant electrode mount 204 may be different from the thickness t of the second compliant electrode mount 208. In the illustrated example, each of the compliant electrode mounts 204 and 208 may be about 3/16 inch to ¼ inch thick.

As shown in FIGS. 2 and 3, a cavity 218 is formed in the inner compliant electrode mount surface 210 of the first compliant electrode mount 204 and another cavity 220 is similarly formed in the second compliant electrode mount 208. The cavities 218 and 220 may be formed in the compliant electrode mounts 204 and 208 to increase the level of compliance of the compliant electrode mounts 204 and 208. In this manner, the compliance of the compliant electrode mounts 204 and 208 or the differential movement between the inner and outer compliant electrode mount surfaces 210 and 212 is substantially controlled by or dependent on the thickness t of the compliant electrode mounts 204 and 208 and the width w of peripheral walls 222 formed by the cavities 218 and 220.

FIG. 4 depicts an example manner in which a person's hand 402 may grip the example compliant biometric sensor handgrip 100 of FIGS. 1 and 2 and apply a plurality of forces to the electrodes 104 and 106 as the person exercises. In particular, the hand 402 may subject the electrodes 104 and 106 to gripping forces indicated by arrows 404a-e and 406a-e and shear forces that are coplanar with the major surfaces (e.g., the surfaces 210 and 212 depicted in FIGS. 2 and 3) of the electrodes 104 and 106 in directions generally indicated by arrows 408a-b. The compliant electrode mounts 204 and 208 may be designed or configured as described above to enable at least portions of the compliant electrode mounts 204 and 208 to deform or displace as shown in FIGS. 5-7 so that the amount of contact or contact area between the hand 402 and the electrodes 104 and 106 is substantially unaffected by the forces indicated by arrows 404a-e, 406a-e, and 408a-b and so that a person's handgrip remains substantially consistent or unchanged as the forces vary over time.

FIGS. 5-7 illustrate representative cross-sectional views of the example compliant biometric sensor handgrip 100 of FIGS. 1, 2, and 4 depicting by way of example some of the manners in which the compliant electrode mounts 204 and 208 may be deformed, moved, or displaced in response to the forces indicated by arrows 404a-e, 406a-e, and 408a-b in FIG. 4. The example compliant biometric sensor handgrip 100 is shown in FIGS. 5-7 with respect to reference points A-D indicating points at which the electrode 104, the compliant electrode mount 204, and the handgrip base 102 are in engagement. Reference points A and B indicate locations at which the inner compliant electrode mount surface 210 engages the handgrip base surface 202. Reference points C and D indicate locations at which the electrode 104 engages the outer compliant electrode mount surface 212.

In FIG. 5, the example compliant biometric sensor handgrip 100 is shown in a neutral state (e.g., no forces are applied to the electrodes 104 and 106), in which reference points A and C are substantially horizontally coplanar. Applying a force (e.g., the force generally indicated by arrow 408a of FIG. 4) to the electrode 104 along the length of the example compliant sensor handgrip 100 and coplanar with the outer compliant electrode mount surface 212 causes the compliant electrode mount 204 to deform by shifting or displacing the inner compliant electrode mount surface 210 relative to the outer compliant electrode mount surface 212 as shown in FIG. 6.

As shown in FIG. 7, applying forces (e.g., the forces generally indicated by arrows 404a-c of FIG. 4) to the electrode 104 toward the handgrip base 102 near the upper portion of the electrode 104, causes the compliant electrode mount 204 to deform by compressing the upper portion of the compliant electrode mount 204 such that the distance between the upper portion of the inner compliant electrode mount surface 210 and the upper portion of the outer compliant electrode mount surface 212 is smaller than the distance between the lower portion of the inner compliant electrode mount surface 210 and the lower portion of the outer compliant electrode mount surface 212.

FIG. 8 is an example elliptical cross-trainer exercise machine 800 having handgrips 802 implemented using the example compliant biometric sensor handgrip 100 depicted in FIGS. 1, 2, and 4. The example elliptical cross-trainer exercise machine 800 includes swinging arm levers 804 on which the handgrips 802 are mounted. During exercise, a person grips the handgrips 802 to move the swinging arm levers 804, which causes the person's hands to apply continuously varying forces (e.g., the forces described above in connection with FIG. 4) to electrodes (e.g., the electrodes 102 and 104 of FIGS. 1, 2, and 4-7) of the handgrips 802. By implementing the handgrips 802 as described above in connection with the example compliant biometric sensor handgrip 100, the person's hands can maintain substantially constant or consistent contact with the electrodes to reduce the amount of noise generated by the electrodes and to improve detection of physiological signals in the person's hands to generate, for example, more consistent and accurate heart rate information.

FIG. 9 depicts an example stair-stepper exercise machine 900 having handrails 902 that include compliant biometric sensor apparatus 904 implemented substantially similar to the example compliant biometric sensor handgrip 100 of FIGS. 1, 2, and 4. Although, the handrails 902 are stationary, unlike the swinging arm levers 804 of FIG. 8, forces applied by a person's hands to the compliant biometric sensor apparatus 904 during exercise may vary due to the person's body motions. However, by implementing the compliant biometric sensor apparatus 904 to include compliant electrode mounts (e.g., the compliant electrode mounts 204 and 208 of FIGS. 2 and 3) as described above, the person's hands can maintain substantially constant contact with electrodes (e.g., the electrodes 102 and 104) of the compliant biometric sensor apparatus 904 to reduce the amount of noise generated by the electrodes and to improve detection of physiological signals via the person's hands.

Of course, compliant biometric sensor apparatus such as the example compliant biometric sensor handgrip 100 of FIGS. 1, 2, and 4 and the compliant biometric sensor apparatus 904 of FIG. 9 may be implemented on exercise machines other than the example elliptical cross-trainer exercise machine 800 of FIG. 8 and the example stair-stepper exercise machine 900 of FIG. 9. For example, compliant biometric sensor apparatus may be implemented on stationary bicycle exercise machines, recumbent bicycle exercise machines, stationary rowing machines, weight training machines, etc. Additionally, compliant biometric sensor apparatus may be implemented in non-stationary apparatus including, for example, outdoor bicycles.

Although certain methods, apparatus, systems, and articles of manufacture have been described herein, the scope of coverage of this patent is not limited thereto. To the contrary, this patent covers all methods, apparatus, systems, and articles of manufacture fairly falling within the scope of the appended claims either literally or under the doctrine of equivalents.

What is claimed is:

1. A biometric sensor apparatus, comprising:
a handgrip having a length and at least one substantially compliant portion extending along at least a portion of the length; and
at least one electrode coupled to the compliant portion so that the electrode is movable relative to at least a portion of the handgrip.

2. An apparatus as defined in claim 1, wherein the compliant portion comprises a rubber material having a first surface engaged to the handgrip and a second surface engaged to the electrode, and wherein the rubber material enables differential movement between the first surface and a second surface.

3. An apparatus as defined in claim 1, further comprising a plurality of electrodes coupled to the compliant portion.

4. An apparatus as defined in claim 1, wherein the electrode is not in contact with a frame portion of the handgrip.

5. An apparatus as defined in claim 1, wherein the electrode is configured to measure physiological signals.

6. An apparatus as defined in claim 1, wherein the handgrip forms a portion of a handrail.

7. An apparatus as defined in claim 1, wherein the compliant portion comprises a material having a Shore D durometer hardness of about 30-35, and wherein the handgrip comprises a material having a Shore D durometer hardness of about 70-80.

8. A biometric sensor apparatus, comprising:
a handgrip base having a first hardness;
an electrode mount coupled to the handgrip base and having a second hardness substantially different than the first hardness; and
an electrode coupled to the electrode mount so that the electrode is substantially movable relative to the handgrip base.

9. An apparatus as defined in claim 8, wherein the first hardness is about 70-80 Shore D durometer hardness, and wherein the second hardness is relatively less hard than the first hardness.

10. An apparatus as defined in claim 8, wherein the handgrip base forms a portion of a handrail.

11. An apparatus as defined in claim 8, wherein the electrode is used to measure a heart rate.

12. An apparatus as defined in claim 8, wherein the electrode mount comprises a rubber material.

13. An apparatus as defined in claim 8, wherein the electrode mount prevents contact between the electrode and the handgrip base.

14. An apparatus as defined in claim 8, wherein the electrode mount has a cavity formed therein.

15. An exercise apparatus, comprising:
at least one handgrip having a base portion, wherein the handgrip includes at least one electrode configured to be substantially movable relative to the handgrip base portion in response to a person gripping the handgrip and the electrode and using the exercise apparatus.

16. An apparatus as defined in claim 15, wherein the handgrip forms part of a handrail.

17. An apparatus as defined in claim 15, wherein the electrode is coupled to the handgrip via a rubber material.

18. An apparatus as defined in claim 15, wherein the electrode is configured to detect physiological signals.

19. An apparatus as defined in claim 15, wherein the handgrip includes a plurality of electrodes configured to be substantially movable relative to the handgrip base portion.

20. An apparatus as defined in claim 15, wherein the base portion is sleeve-like and engaged to a tubular portion of the exercise apparatus.

21. A biometric sensor mounted to an exercise apparatus, comprising:
a pliable member having a first surface engaged to the exercise apparatus, wherein a characteristic of the pliable member enables differential movement between a first surface and a second surface of the pliable member; and
a conductive element engaged to the second surface.

22. A biometric sensor as described in claim 21, wherein the conductive element is configured to detect physiological signals.

23. A biometric sensor as described in claim 21, wherein the conductive element is communicatively coupled to a system configured to generate biometric information.

24. A biometric sensor as described in claim 21, wherein the pliable member comprises a rubber material.

25. A biometric sensor as described in claim 21, wherein the pliable member is engaged to a handgrip surface of the exercise apparatus.
